(19) United States
(12) Patent Application Publication
(10) Pub. No.: US 2005/0137205 A1
(43) Pub. Date: Jun. 23, 2005

(54) Formulation for treating periocular disease

(76) Inventor: Eduard T. Van Breen, Manchester, VT (US)

Correspondence Address: Arthur R Eglington, 601 Stoney Run Road, R.D.#5, Pottsville, PA, 17901, US

(21) Appl. No.: 10/742,149

(22) Filed: Dec. 22, 2003

(51) Int. Cl. A61K 31/495; A61K 31/4164; A61K 31/198

(52) U.S. Cl. 514/252.12; 514/389; 514/562

(57) ABSTRACT

A composition and method which combines, in one singular therapeutically effective application, antiinflammatory properties with cell proliferant and tissue repair qualities, together with cleansing properties for the periocular skin. The composition includes, in approximate parts by weight, Deionized water (20-80 by composition) Sodium Lauryl Sulfate in concentration of 0.3-20, Potassium C12-13 Monoalkyl Phosphate Polysorbate 60 (0.5-15), Potassium C-12-13 Monoalkyl phosphate (0.5-15), Disodium Lauroamphodiacetate (0.02-12), Linoleamidopropyl PG-Diammonium Chloride Phosphate (0.01-8), Sodium Chloride (0.1-6, HEPES Acetate (0.01-7.7), Citric acid (0.5-7.5) Diazolidinyl Urea Propylene Glycol/Methyl-Propyl Parabens (0.01-5.7), Panthenol (0.01-10), Glyoxyl-Diureide/Allantoin (0.01-15), Polysorbate 80 (0.01-5), and Fragrance (0.005-3). The composition also contains a zwitterionic-fatty acid compound having anti-inflammatory properties containing an effective amount of a skin-compatible component with the compound being an ester, ether, urethane, amide or urea of one HEPES compound.

This is an examinable patent application under Patent Code Section 111 (a) submitted for a formal filing receipt and subsequent examination.

### FIELD OF INVENTION

This invention relates to the most common eye disease in the USA, “Dry Eye”, which can be defined as the lack of quantity and quality of the tear film.

This invention takes into account that periocular skin has very distinct characteristics from other parts of the largest human organ, the skin. It also takes into account the relationship between periocular skin and “dry eye.” “Dry Eye” disease may lead to discomfort, and when left untreated, leads to loss of vision.

### BACKGROUND OF INVENTION

A tear film is critical for comfort and vision. A lack of tears results in the lack of nutrients and oxygen to the cornea. Dry Eye is the most common eye disease in the U.S. and afflicts nearly a third of the population. The disease is chronic and has no known cure.

This disease may lead to discomfort and, when left untreated, the consequences can lead to loss of vision. (e.g. “Desiccation of the corneal epithelium, ulceration and perforation of the cornea, an increased incidence of infectious disease and potentially, serious impairment and blindness”) There are two types of Dry Eye Syndromes: Tear-deficiency Dry Eye and Evaporative Dry Eye.

The former can be divided, whether it is related to the “Sjögren Syndrome” or not. Sjögren syndrome is a chronic disease in which white blood cells attack the moisture-producing glands. Non-Sjögren Syndrome related type of Dry Eye symptoms do not produce tears because of diseases of the tear gland. Examples of causes are eye infections, vitamin A deficiency, contact lens wear and diabetes. The possible causes of Evaporative Dry Eye Syndrome include skin disorders such as Blepharitis, Rosacea, Meibomian Gland Dysfunction (all peri-ocular related issues). Other causes are ocular surface diseases and environmental factors such as contact lens wear, dust, smoke, debris, air-conditioning. Also eyelid disorders. Such as proptosis, ectropion, entropion, incomplete blinking, pterygia pingueculae, conjunctivochalasis, nocturnal lagophthalmos.

The direct relationship between the condition of the skin around the eyes and our ability to see is too often trivialized, or overlooked, even though this area significantly contributes to the normal physiology and functioning of the ocular surface on a constant basis, for the reason that all the tear producing glands are not located in the cornea, but outside the eye surface in the periocular skin and conjunctiva.

It is critical, therefore, to appreciate that the periocular skin is distinct from other parts of the skin. The differences are notably that this area contains ten times less lipid in the corneum stratum, that the corneum stratum has fewer layers, that it has higher epidermal kinetics and that it is located close to a warm and moist environment. Current approaches in dealing with Dry Eye are focused on treatment. Current main methods are artificial tears, antibiotics, punctum plugs (tiny plugs that prevent draining of the tear film into the nasal cavity), some nutritional supplements and even special goggles called “moisture chamber spectacles”. While the root problem of Dry Eye is related to eyelid disease, eyelid hygiene is only prescribed after irritation occurs, or after encrustation of the eyelids, or after other lid diseases are diagnosed such as Blepharitis, Sty, Rosacea, external hordeolum and inflammation of the eye lids. The most common form of lid cleansing solution prescribed is simple “baby shampoo”. Another type of product for eye lid cleansing is commonly referred to as “lid scrubs” or “eye scrubs,” or simply as “non-irritating” liquid cleansing solutions.

Current research underscores that the physiological processes in and on the skin take place best in a slightly acidic medium. To support the skin's functions, it is therefore important to use skin care products with a physiological pH ideally close to the skin's pH of 5.5. In addition, it is important to stabilize the skin's natural protective acid mantle to optimize its ability to fend of infections.

Taking into account the fact that the periocular skin already has ten times less lipid and fewer layers in the outer corneum stratum, emulsification of this critical oily compound should best be avoided, since it will increase the chances for creating pathways for pathogens and skin irritation.

In addition, the rate of transdermal water loss from the skin is directly related to the damage of the stratum corneum. Surfactants, (cleansing properties) are often the cause of much of the damage.

While daily eyelid hygiene is critical in the fight against infections, with a cleansing solution that takes into account the many distinct characteristics, optimization and swift repair of damaged tissue of this unique periocular skin is equally essential and beneficial.

To be able to address the tissue repair of the fragile peri-ocular skin, one needs to take into account that many skin disorders, such as seborrheic dermatitis, eczema, bacterial and viral infections, fungal infections, contact dermatitis, and acne are related to the dermatological mechanism of inflammation.

Currently, there does not exist a product that incorporates s; the special hygiene requirements of the periocular skin with a complex of ingredients that would help in tissue repair, in optimization and stimulation of the overall health of the orbital area, or aid in addressing the age related changes of this particular skin (which is in constant motion). A complex that is gentle enough to be used on a daily basis and which would thus optimize the environment and ability for the tear producing glands to function.

Current products only contain cleansing components and no skin improvement properties, and/or lack anti-inflammatory components and/or deliver the solution in aggressive alkaline pH levels, and/or are difficult to administer and could possibly damage the skin's surface.

### DESCRIPTION OF PRIOR ART

Prior patents below have in common that none of them contain anti-inflammatory agents selected from the zwittererionic organic compounds. Also these prior patents share that they do not include beneficial tissue repair properties stemming from Allantoin, or Panthenol, or Citric Acid.

Some prior patents that address the health of the periocular skin have in common the draw back that they address only one, or two particular aspects of the complex needs of the distinct periocular skin, rather than combining cleansing, treatment, prevention, maintenance, and compliance issues relating to the periocular skin in one single, application.

By way of examples, U.S. Pat. No. 5,252,246 is only to a nonionic surfactant composition and U.S. Pat. No. 5,000,868 is to another surfactant, through a combination of nonionic and anionic surfactants, with an amine oxide surfactant accelerator. Some existing cleansing solutions, or ointments, or surfactant composition or antibiotics seem to overlook the fragile nature of the eye contour. They have a high alkaline pH that is considerably higher than the acid pH of the skin, seemingly overlooking the nature of the periocular skin which contains already less lipid and emulsification of these lipids in the corneum stratum need to be minimized, especially also considering that the corneum stratum of this periocular skin has significantly less layers than elsewhere in the skin. Example; Patent 4904698 claims to need a pH of 8.0-8.5, which is nearly 1000 times more alkaline than the skin.

On the other hand U.S. Pat. No. 6,573,299 describes a method and composition for treatment of aging eye by using an aggressive acid composition by suggesting the use of Alpha Hydroxy Acid and or Beta Hydroxy Acids. The introduction of such acids (their suggested pH is 34 or nearly twice as acid as the periocular skin) close to the eye may result in further complications, but also to introduce such exfoliating properties to the thin periocular skin overlooks the very nature of this part of the skin, namely that the periocular skin not only exfoliates already twice as fast as other parts of the skin, but that it also has many less layers in the corneum stratum. To accelerate the natural kinetics of this part of the skin may lead to yet more additional complications. For example, by exposing the epidermis to the environment with an immature corneum stratum and leading to pathways for pathogens.

U.S. Pat. No. 6,455,583 is not a cleansing solution and skin treatment composition, but is an antibiotic treatment composition for the meibomian gland dysfunction through use of tetracycline, or a derivative of tetracycline, while U.S. Pat. No. 4,612,193 describes a sty ointment based on anti bacterial properties of mercuric oxide using boric acid as a stabilizer, in an emollient, to be topically applied to the eyelids and to be left as a thin coating on the lid margin or inflamed areas.

### OBJECT OF THE INVENTION

It is an object of the invention to address the specific disorders of the skin, which are related to the mechanism of inflammation.

It is another object of the invention to also help optimize the health of the epidermis and dermis in the same formulation.

It is the object of the invention to be non-irritating to the eye.

It is yet another object of the invention that the product will be even less disturbing to the skin than baby shampoo.

It is an added object of the invention to have a special pH for the overall formulation, closer to the 5.5 pH of the skin.

It is yet another objective of the invention to minimally disturb the stratum corneum, the lipid layer and minimize transdermal water loss.

It is the objective of the invention to improve the tear breakup time, which is the measurement of dry eye, and to contribute to the increase in comfort of patients.

### SUMMARY OF THE INVENTION

The present invention offers a multifaceted complex that combines in therapeutically sufficient strength, special peri-ocular skin cleansing properties with unique tissue repair, treatment and anti-inflammatory ingredients in a single application. It optimizes the physiological process of the skin by having an acid medium similar to that of skin and is yet not irritating to eyes or skin.

The presently preferred composition includes Deionized water (20-80% by composition) Sodium Laureth Sulfate in concentration of 0.3-20%, Potassium C12-13 Monoalkyl Phosphate Polysorbate 60 (0.5-15%), Potassium C-12-13 Monoalkyl phosphate (0.5-15%), Disodium Lauroamphodiacetate (0.02-12%), Linoleamidopropyl PG-Diammonium Chloride Phosphate (0.01%-5.0%), Sodium Chloride (0.1-6%), HEPES Acetate (0.01-7.7%), Citric acid (0.5-7.5%) Diazolidinyl Urea Propylene Glycol/Methyl-Propyl Parabens (0.01-5.7%), Panthenol (0.01-10%), Glyoxylic-Diureide/Allantoin (0.01-15%), Polysorbate 80 (0.01-5%) and a fragrance (0.005-3%). The complex may be self administered on a daily basis, or as frequently as is required.

In a preferred embodiment of the invention, the following active ingredients are usefully combined as having proven clinical performance as an eyelid cleanser, as described in the objects of the present invention: HEPES, oleate ester as an anti-inflammatory agent (2.00); a pH-control agent citric acid (1.50); a first tissue healing agent allantoin (0.25); a first water soluble surfactant potassium C12-13 monoalkyl phosphate (5.0); a second water soluble surfactant disodium lauroamphodiacetate (4.0); a preservative diazolidimyl urea propylene (1.00); a skin conditioning and antibacterial phospholipid essential fatty acid (0.1-5.0); and a viscosity regulating sodium chloride (7.00). Optionally, for ascetic reasons, a natural fresh and clean fragrance may be included in the preferred formulation.

The present invention recognizes the relationship between the most common eye disease in the United States (“Dry Eye”) and the area were most of the critical tear producing glands are located. The invention focuses on the distinct differences between periocular skin and ‘normal’ skin and addresses among other requirements of the periocular skin, the special low lipid content, the lower layer count in the corneum stratum, its overall fragility, its higher rate of exfoliation, and the warm and moist environment which is conducive for infections.

The invention further recognizes that skin disorders are related to the mechanism of inflammation.

### PREFERRED EMBODIMENT OF THE INVENTION

The periocular skin is distinct in nearly every important function from the rest of the body and would require special attention, especially since it already is the first to manifest age related changes. At the same time, this distinct periocular skin also provides accommodation for most all of the important tear producing glands that are responsible for our tear film. (e.g The Meibomian Glands, the glands of Zeiss, Moll, Kraus).

Optimization of these glands is critical in addressing Dry Eye. By definition then, a very special approach is required to the periocular skin, not in the least because it is touching the warm and moist eye, or is close to the eye, representing an increased risk for infections.

Optimization of the area around the eyes, must include daily peri-ocular hygiene, and because this skin is very distinct and extremely fragile, optimization should also include methods to address skin disorders, such as viral and bacterial infections. By way of illustration, bacterial infections are largely determined by the ability of bacteria to invade the skin (as well as the relative toxicity of the bacteria). Considering that the skin around the eyes is substantially thinner, but also that it contains less lipids, we would wish to ensure that we don't wash away those lipids and thus more easily create pathways for bacteria to enter. Considering it is in constant motion and most fragile, but also because it is close to the warm and moist eye, combined with the fact that many of the causes of Dry Eye are chronic, with no known cure, rapid and continuous tissue repair of the periocular skin is critical.

Our invention represents a multifaceted composition that incorporates cleansing, tissue repair, preventive maintenance and treatment properties in a singular application, in therapeutically effective amounts which allows for frequent daily, self-administration as needed. The complex specifically takes into account the distinct characteristics of the periocular skin, such as, but not limited to, the lower lipid count, the thinner and lower count in stratum corneum layers, the higher rate of exfoliation, the age-related changes and the fact that it represents the arena where most all the tear producing glands are located.

The complex recognizes these special needs of the periocular skin and its direct relationship with the epidemic eye disease “Dry Eye”. Daily optimization of the tear producing glands is critical especially given that daily the quantity and quality of performance of these glands diminish. Dry Eye is an age related disease. Optimization of the tear producing glands, must include eye hygiene, since debris may not only help block the tear ducts, it may lead to a breathing ground of pathogens close to the warm and moist environment that the eye represents. Especially since Meibomian Gland Dysfunction is the most frequent infection in Dry Eye and manifests itself in such forms as encrustation of the lid margins, sty, hordeolum or other inflammation of the connective tissue.

The present invention is not irritating to the eye. The acid formulation of the invention, having a pH close to the pH of the skin, minimally disturbs the lipid content and helps allow for frequent application. The acid formulation also optimizes the skin's physiological recovery abilities. Moreover it has been proven to be less disturbing to the skin than the standard hypoallergenic baby shampoo.

Meibomian gland Dysfunction, the major cause of Dry Eye, is commonly linked with Ocular Rosacea and Blepharitis, and other inflammation of the eyelids. It has been shown that frequent daily use of this invention significantly improves the tear break up time, (TBUT is the measurement of Dry Eye) in Blepharitis and Rosacea patients.

Among the commercially available water-soluble surfactants useful with this invention are: sodium lauryl sulfate (CALFOAM ES 303); potassium C12-13 monoalkyl phosphate 60 (ARLATONE MAP 230-T60); C12-14 monoakyl phosphate (ARLATONE MAP 230K40); disodium lauroamphodiacetate (MONATERIC 949-J); and linoleamidopropyl PG-chloride phosphate (PHOSPHOLIPID EFA). Special ingredients such as Allantoin, Panthenol and Citric Acid are encapsulated in the cleansing solution so that the skin is in better shape after washing than before.

The present invention employes HEPES derivatives which are pharmacologically active as anti-phospholipase and anti-inflammatory compounds specifically, wheren the active ingredients are certain long chain esters of selected zwitterionic compounds, based on an N-substituted taurine, namely aliphatic esters of HEPES. Generally, HEPES, and its homologs are N-(2-hydroxy ethyl)-piperazine-N′-(2-alkane sulfonic acid).

Generally, an alkali metal salt of HEPES is catalytically reacted with an alkyl-substituted, either saturated or unsaturated, aliphatic salt, such as methyl oleate, methyl linoleate, methyl palmitate, methyl stearate, methyl myristate, and methyl behenate. They are reacted in equimolecular amounts, carried out either with or without a non-aqueous solvent, such as acetone, and in a temperature range of 0° C. to variable degrees C.; which is between 0° C. and the chosen solvent's reflux temperature. The purification of the crude ester is carried out by means of crystallization in an organic solvent, dissolved in methanol, and recrystallized. The U.S. Pat. No. 6,114,337, granted Sep. 5, 2000, provides detailed synthesis examples of the zwitterionic organic compounds useful here, including the ether analog and urethane derivatives of HEPES.

A prior art formulation for Dry Eye is set forth in Table I.

### EXAMPLE 1

The cleansing solutions of the present invention have the below-listed range of physical parameters to be most useful.

Specifications:

| pH | 5.5 to 5.7 |
|---|---|
| Specific Gravity | 1.070 +/− 0.0005 |
| Viscosity | >700 cps +/− 0.5%% |
| Appearance: | Sl. Cloudy, concentrated bubbly gel |
| Plate Count: | 0 |

The lid cleansing solution of Working Example II (Table II) was tested at a 10% concentration in water to represent normal dilution with tap water. The purpose: To determine the ocular irritation potential of the test material following instillation into the eyes of six rabbits when tested in a 10% (v/v dilution in USP Water for Irrigation).

Test System: New Zealand albino rabbits, male and female, weighting between 2.54. and 2.87 kg. Six per test group.

Method: Prior to administration, both eyes of each of six rabbits were examined for injury. The material was diluted to 10% in USP Water for Irrigation, then 0.1 ml was instilled into the lower conjuncival sac of one eye of each of six rabbits. The opposite eye of each animal remained untreated and served as control. Observations were performed at 24, 48 and 72 hours for signs of opacity, iritis and conjunctivitis.

Scale for scoring ocular lesions:

| | |
|---|---|
| 1-Cornea (area of opacity most dense taken fro reading) | |
| No Opacity | 0 |
| Scattered or diffuse area, details if iris clearly visible | 1 |
| Easily discernible translucent areas, details of iris slightly obscured | 2 |
| Opalescent areas, no details of iris visible, pupil size barely | 3 |
| discernible Opaque, iris invisible | 4 |
| 2-Iris | |
| Normal | 0 |
| Folds above normal, congestion, swelling, circum corneal injection | 1 |
| (any or all of these or combination of any thereof) iris still reacting | |
| to light(sluggish reaction is positive) | |
| No reaction to light, hemorrhage, gross destruction | 2 |
| (any or all of these) | |
| 3-Conjunctivae | |
| A Redness(refers to palpebral and bulbar conjunctivae | |
| excluding cornea and iris) | |
| Vessels normal | 0 |
| Vessels definitely injected above normal | 1 |
| More diffuse, deeper crimson red, individual vessels not easily | 2 |
| discernible | |
| Diffuse beefy red | 3 |
| B. Chemosis | |
| No Swelling | 0 |
| Any swelling above normal (including nictitating membrane) | 1 |
| Obvious swelling with partial eversion of lids | 2 |
| Swelling of lids about half closed | 3 |
| Swelling with lids about half closed to completely closed | 4 |
| C. Discharge | |
| No discharge | 0 |
| Any amount different from normal (does not include small amounts | 1 |
| Observed in inner canthus of normal animals) | |
| Discharge with moistening of lids and hairs just adjacent to lids | 2 |
| Discharge with moistening of lids and hairs, and considerable area | 3 |
| Around the eyes | |

Definition
R = Redness with grades of 0, 1, 2*, 3*
CH = Chemosis with grades of 0, 1, 2*, 3*, 4*
I = Iritis with grades of 0, 1*, 2*
Corn = Corneal opacity with grades of 0, 1, 2*, 3*, 4*
*= positive score:
RESULTS
“0” (zero) results were found in each of tested animals, for each of the tested parameters, for each of the period of duration (day 1, 2 and 3).

### IN SUMMARY,

1. no irritation was observed in the eyes of rabbits following administration
2. all animals appeared healthy throughout the course of the study
   Conclusion

Under the conditions of this test, the test material was not considered a primary eye irritant.

### Clinical Example Two

The effect of eyelid cleanser of Working Example II (Table II) on the Tear Break Up Time in Ocular Rosacea—associated Meibomian Gland Dysfunction

Objective:

To determine whether daily lid hygiene with a proprietary lid cleanser, has a significant effect on the tear break up time (TBUT) in patients with meibomian gland dysfunction (MGD) secondary to ocular rosacea.

Methods:

Two sub-groups of patients with ocular rosacea (a total of 35 patients) were identified and placed on a regimen of daily eye hygiene on one eye, and hypoallergenic baby shampoo on the other. Both groups were also given doxycycline 100 mg po bid for 60 days. Group 1 had foam/froth present a the meibomian orifices, and/or foamy/frothy tears. Group 2 did not exhibit foaming or frothing at the gland orifices or in the tear film. Cleansing solutions were randomized between right and left eyes, placed in non-marked plastic dispenser bottles labeled Right and Left, and neither patient nor examiner was informed which solution was which. Lid cleansing was performed bid, AM & PM according to instructions given. Patients' symptoms, slit-lamps findings and TBUT were determined at 30, 45 and 60 days.

Results:

At the end of 60 days, a significant difference between the proprietary lid cleansing solution and the baby shampoo was noted in both groups. In patients presenting with foam/frothy tears (N-19) and increase in TBUT in the proprietary lid cleansing solution of +3.49 seconds versus a smaller increase of +1.53 seconds for the eyes treated with baby shampoo was noted. In the non-foamy/frothy tear film group (N=16), the proprietary lid cleansing solution was determined to increase TBUT by +3.03 seconds compared to +2.17 seconds for baby shampoo.

Conclusions

The proprietary lid cleansing solution, significantly improved the quality of the tear film in patients with ocular rosacea and meibomian gland dysfunction as indicated by an increase from baseline of the tear breakup time when compared to generic hypoallergenic baby shampoo in a masked trial of both cleansers.

### Clinical Example Three

The effect of the proprietary eyelid cleanser of Working Example II (Table II) on the TBUT in Chronic Blepharitis.

Objective: To determine whether the present proprietary eye hygiene solution has a significant effect on the TBUT in patients with posterior Blepharitis/Meibomianitis

Methods:

Various sub-groups of patients with chronic (>6 months) Blepharitis were identified and placed on a regimen of the proprietary eye hygiene solution on one eye, and hypoallergenic baby shampoo on the other. Cleansing solutions were randomized between right and left eyes, placed in non-marked plastic dispenser bottles labeled Right and Left, and neither patient nor examiner was informed which solutions was which. Patients were instructed to perform lid cleansing twice per day, once in the AM and once in the PM prior to retiring for the night. Patients symptoms, slit-lamp findings and TBUT were determined at 30, 45 and 60 days.

Results:

At the end of 60 days a significant difference between patients presenting with MGD was observed with regards to the TBUT in the eyes using the proprietary eye hygiene solution versus baby shampoo. Patients with MGD and a frothy/foamy tear film (N=22) showed an overall increase in TBUT of +4.03 seconds versus a decrease of −0.57 seconds for the baby shampoo cohort of eyes. Patients with MGD and non-frothy tear film (N=15) exhibited an increase in TBUT of +2.83 seconds in the proprietary eye hygiene solution versus only a slight increase of +0.39 seconds for the eyes treated with baby shampoo.

Conclusions

The proprietary eye hygiene solution, a non-soap product, significantly improved the quality of the tear film in patients with posterior Blepharitis/meibomian gland dysfunction as measured by the tear break up time (TBUT) when compared to generic hypoallergenic baby shampoo in masked trial of both cleansers.

### Clinical Example Four

Study to determine the effects of the present eye hygiene solution and baby shampoo on the keratin proteins of the stratum corneum.

Method:

The test is based on the principle of denaturation of protein denaturation by surfactant systems. It is well known that surfactants adversely affect protein structures and can remove lipids for the stratum corneum. The method used in this study is based on the acridine orange staining of the stratum corneum, a florescent stain to detect biopolymers. This sensitive technique proves a means for qualitative and quantitative comparison and correlation between data obtained from solutions and from data obtained from cells. When acridine orange is in a concentration in excess of 2×10-6 M the dye molecules will interact with each other and produce metachromasia. The monomer of the dye emits a green florescence with a peak at 540 nm. The dimmer and higher polymers emit a red fluorescence with a peak a t660 nm. The color emitted depends on the nature of the biopolymer and the dye-to polymer ratio. Glycosaminoglycans emit red fluorescence with AO, except hyaluronic acid which emits green florescence. Nucleic acids emit green at times red fluorescence. One milliliter of the test products at full strength was placed on the volar surface of the forearm of the test subject covering 16 cm2 area. The products remained on the arm for fifteen minutes. One milliliter of tap water placed on the same surface to act as a control. The test sites were sampled with a drop of cyanoacrylate glue on a standard microscope slide. The slides were slides were stained with 0.1% acridine orange solution pH 6.8 for 15 minutes and examined with a 40 power and 100 power fluorescence microscope.

Results:

The water treated skin served as the control slide which the pattern of the surface markings and the colors were obtained as a standard for undamaged skin. Noted were the regular patterns of the markings the metachromasia and the cellular detail between the markings, or ridges. The baby shampoo treated skin showed marked deterioration of the marking with an unraveled appearance of the keratin. Cellular detail is distorted and obscured in most areas.

The proprietary eye hygiene cleanser treated skin showed only mild to moderate changes in the surface pattern compared to the control, or water treated skin.

Conclusion

Based on the study it is concluded that the proprietary eye hygiene cleanser is less damaging to skin than baby shampoo. The denatured protein would suggest a damaged stratum corneum that would be more permeable to water under the eye lid and could contribute to dry eye syndrome. The rate of trans-epidermal water loss from the skin is directly related to the degree of damage in the stratum corneum.

In summary, it has been established that a special cleansing treatment solution for the periocular skin can be formulated to the benefit of patients, that takes into account the unique and distinct characteristics of the skin surrounding the eye, and the eyelid. Not only is the invention not irritating to the eye, it disturbs the skin less than hypoallergenic baby shampoo. The invention recognizes that most all ophthalmic skin disorders, such as viral and bacterial infections are related to the dermatological mechanism of inflammation. The invention introduces unique anti-inflammatory properties, while encapsulating tissue repair and treatment benefits as well. Overall the transdermal water loss and major violation of the stratum corneum is minimized and the physiological recovery ability of the skin is optimized through an acidic formulation with a pH close to the ph of skin, while being non-irritant to the eyes. The invention recognizes that the special skin surrounding the eye is the first to show age related changes, because it is more fragile, yet thinnest and in constant motion. The invention recognizes the special task that the skin needs to perform, such as protecting the eye surface, spreading of the critical tear film and housing the tear producing glands, whereby health boosting properties are introduced. In this manner, the disclosed cleansing treatment formulation enables the skin to be in better shape after each cleansing than before. This invention has shown further that it has a significant positive contribution to the Tear Break Up Time.

The invention in its broader aspects is not limited to the specific details shown and described, but departures may be made from such details, within the scope of the accompanying claims, without departing from the principles of the invention and without sacrificing its advantages.

TABLE I

| % | PRIOR ART FORMULATION |
|---|---|
| 69 | Aqueous extract of chamomile |
| 0 | |
| 14 | Potassium C-12-13 monoalkyl phosphate 80, |
| | Polysorbate 80 |
| 5 | Potassium C12-13 monoalkyl phosphate |
| 5 | Disodium Lauroamphodiacetate |
| 4 | Sodium Borageamidopropyl hydroxyphosphate |
| 1 | Sodium chloride |
| 0 | |
| 0 | |
| 1 | Diazolidinyl urea |
| | Methlyparaben |
| | Propylparaben |
| 0.5 | Panthenol |
| 0.25 | Glyoxylic-Diureide/Allantoin |
| 0.1 | Polysorbate 80 (a polyoxyethylene fatty acid |
| | ester based on sorbitol) |
| 0 | |
| 99.85 | |

TABLE II

| TRADE NAME | CHEMICAL NAME | FUNCTION | PREF. |
|---|---|---|---|
| DI WATER | Deionized water | Aqueous solution | 60.50 |
| | | Aqueous with soothing properties | 0.00 |
| CALFOAM ES 303 | Sodium Lauryl Sulfate | Surfactant cleanser | 15.00 |
| ARLATONE MAP 230- | Potassium C12-13 | Surfactant cleanser | 5.00 |
| TWEEN 60 ™ | monoalkyl phosphate | | |
| | Polysorbate 60 | Surfactant cleanser | 7.00 |
| ARLANTONE MAP | Potassium C12-13 | Surfactant and lipid replacement | 4.00 |
| 230K40 | monoalkyl phosphate | agent | |
| MONATERIC 949-J | Disodium Lauroamphodiacetate | Surfactant and lipid replacement | 1.00 |
| | | agent | |
| PHOSPHOLIPID EFA | Linoleamidopropyl PG- | Skin conditioning and antibacterial | 2.00 |
| (essential fatty acid) | Diammonium | agent | |
| | Chloride Phosphate | | |
| SODIUM CHLORIDE | Sodium Chloride | osmolarity controlling agent | 2.00 |
| HEPES, ACETATE ester | N(2-hydroxy ethyl) piperazine-N′- | Antioxidant and antiinflammatory | 1.50 |
| | (2-propane sulfonic acid), | | |
| | acetate salt | | |
| CITRIC ACID | Citric acid 50% Solution | Collagen building agent, antioxidant | 1.00 |
| | | and pH control agent | |
| GERMABEN 11 | Diazolidinyl urea | Optional microbiocidal | 0.50 |
| | Methylparaben | preservative | |
| | Propyl paraben | | |
| PANTHENOL | Panthenol-alcohol analog of | Wound healer, tissue repair & | 0.25 |
| | pantothenic Acid | healing, stimulator of cellular | |
| | | proliferation, antiinflammatory | |
| ALLANTOIN | Glyoxylic-Diureide | Stimulates new and healthy tissue | 0.25 |
| | | growth, healing epithelization | |
| | | Counter irritant, moisturizer, softens | |
| | | skin | |
| TWEEN 80 ™ | Polysorbate 80 (fatty acid ester | Solubilizer of optional fragrance | |
| | based on sorbitol) | inclusion | |
| | | | 100.00 |

1. An antiinflammatory, aqueous composition for treating periocular skin comprising as active ingredients in approximate parts by weight:
(a) an antiinflammatory agent in the range of 0.01 to 7.7, selected from the zwitterionic organic compounds group consisting of essentially aliphatic long chain esters synthesized from piperazine-N′ propane sulfonic acid;
(b) a pH-control agent and antioxidant agent in the range 0.05-7.5 selected from the group consisting of carboxylic acid derivatives of propane;
(c) a first tissue healing agent in the range of 0.01 to 1 selected from the group consisting of allantoin and panthenol;
(d) a first water soluble surfactant present in the range 0.02-20 selected from the group consisting of potassium C12-13 monoalkyl phosphate polysorbate 60 and potassium C12-13 monoalkyl phosphate;
(e) a second water soluble surfactant in the range of 0.01 to 7.0 which also aids in lipid replacement selected from the group consisting of disodium lauroamphodiacetate and linoleamidopropyl PG-diammonium chloride phospate;
(f) a skin conditioning and antibacterial agent comprising a phospholipid essential fatty acid in the range of 0.1.to 5.0;
(g) a microbiological preservative present in the range of 0.01-5.7 and selected from the group consisting of phenoxyethanol and diazolidinuyl urea propylene glycol/methyl-propyl parabens; and,
(h) a viscocity regulating agent in the range of 0.1 to 6 selected from one of the alkali metal salts of hydrochloric acid.

2. The composition of claim 1 wherein the active zwitterionic organic compound is a long chain aliphatic ester of N-(2-hydroxylethyl)piperazine N′(12-propane sulfonic acid) (HEPES).

3. The composition of claim 2 wherein the active zwitterionic organic compound is the acetate ester of HEPES.

4. The composition of claim 2 wherein the long chain aliphatic ester is the oleate ester of HEPES.

5. The composition of claim 2 wherein the active zwitterionic organic compound is the linoleate ester of HEPES.

6. The composition of claim 1 wherein the active zwitterionic organic compound is an ether analog of HEPES.

7. The composition of claim 1 wherein the active zwitterionic organic compound is a urethane derivative of HEPES.

8. The composition of claim 1 wherein a solubilizer is provided in the range of 0.01 to 5 comprising polyoxyethylene derivative of a fatty acid ester of Sorbitol.

9. The composition of claim 1 wherein the first surfactant may contain sodium laureth sulfate in a concentration of 0.3-20.

10. The composition of claim 1 wherein a second preservative is selected from the group consisting of diazolidinyl urea propylene glycol (0.01-5.7), methyl-propyl parabens (0.01-5.7).

11. The formulation as described in claim 1 wherein the pH range is 5.5-6.5.

12. The composition of claim 1 wherein a third surfactant is sodium borage-amidopropyl hydroxyphosphate.

13. The aqueous composition of claim 1 wherein the pH control agent is citric acid (2-hydroxy-1,2,3-propane tricarboxylic acid) providing a pH range of 0.5 to 0.75.

14. An antiinflammatory aqueous composition for treating periocular disease in approximate parts by weight comprising:
(a) a long chain aliphatic ester of N′(2-hydroxy ethyl)piperazine N′-2-propane sulfonic acid in the range of 0.01 to 7.7;
(b) a pH control and antioxidant agent citric acid in the range of 0.05 to 5.0;
(c) a first tissue healing agent allantoin in the range of 0.01 to 1.0;
(d) a second tissue healing agent panthenol in the range of 0.01 to 10; a surfactant lipid replacement agent phospholipid essential fatty acid;
(e) a microbiological preservative comprising diazolidinylurea and a viscosity regulating agent sodium chloride in the range of 0.1 to 6.0;
(f) a surfactant cleanser potassium C12-13 monoalkyl phosphate; and,
(g) a skin conditioning agent comprising the phospholipid EFA linoleamidopropyl PG-diammonium phosphate in the range of 0.5 to 3.0.
